(19) United States
(12) Patent Application Publication
(10) Pub. No.: US 2024/0252575 A1
(43) Pub. Date: Aug. 1, 2024

(54) ORAL HYDROGEL WOUND DRESSING

(71) Applicant: Forward Science Technologies, LLC, Stafford, TX (US)

(72) Inventors: Brian Pikkula, Sugar Land, TX (US); Katherine R. Adkins, Stafford, TX (US); Robert J. Whitman, Houston, TX (US); Kathleen O. Hotze, Houston, TX (US)

(73) Assignee: Forward Science Technologies, LLC, Stafford, TX (US)

(21) Appl. No.: 18/043,829

(22) PCT Filed: Sep. 3, 2021
(86) PCT No.: PCT/US2021/049067

§ 371 Date: Mar. 2, 2023

Related U.S. Application Data
(60) Provisional application No. 63/075,068, filed on Sep. 4, 2020.

(51) Int. Cl. A61K 36/54; A61K 9/00; A61K 9/06; A61K 36/53; A61K 36/61; A61K 36/906; A61K 47/02; A61K 47/36; A61P 17/02

(52) U.S. Cl. CPC A61K 36/54; A61K 9/006; A61K 9/06; A61K 36/53; A61K 36/61; A61K 36/906; A61K 47/02; A61K 47/36; A61P 17/02

(57) ABSTRACT

The invention provides a composition in the form of a hydrogel useful for treating wounds in the oral cavity. The hydrogel includes one or more polysaccharides, one or more polyols, one or more essential oils, and water, and has a pH of from about 6.2 to about 8.5. The invention also provides a method of treating a wound in the oral cavity by applying an effective amount of the composition to the wound.

### CROSS-REFERENCE TO RELATED APPLICATIONS

This patent application is the U.S. National Stage of International Patent Application No. PCT/US2021/049067, filed Sep. 3, 2021, which claims the benefit of U.S. Patent Application No. 63/075,068, filed Sep. 4, 2020, the disclosures of which are incorporated herein by reference in their entireties for all purposes.

### BACKGROUND OF THE INVENTION

In dental practice, medical professionals are often faced with the need to treat wounds in the oral cavity. Trauma-related oral wounds are encountered frequently in clinical dental practice. Wounds in the oral cavity can impair a patient's normal oral function, and cause pain in a patient's eating, chewing, and speaking. An injury to the oral mucosa can result from physical, chemical, or thermal trauma. Such injuries can result, for example, from accidental tooth bite, hard foods, sharp edges of the teeth, hot food, excessive tooth brushing, and exposure to chemicals that are damaging to oral tissues. Some injuries also can be caused by iatrogenic damage during dental treatment, surgery, or other procedures performed within the oral cavity. Most traumatic oral wounds such as, e.g., sores, lesions, or ulcers in the oral cavity, may heal on their own without complication. However, in some cases persistent traumatic factors such as, for example, sharp tooth morphology, sharp edges on dental restorations, and puncturing appliance contours, e.g., from inadequate prosthetic surfaces, can cause continuous trauma which can lead to formation of chronic ulcers.

Common strategies for treating oral wounds include pain relief, infection control, and acceleration/promotion of wound repair. The most common for treating oral wounds is pain relief. Topical dressings are among the preferred protocols for oral wound therapy, as they have considerably lower risk of adverse effects than other protocols such as systemic drugs, surgical excision, and laser treatment. Indeed, some sources have reported that over 30% of all opioids are prescribed by dentists and oral surgeons. By providing a safer alternative for pain management, topical dressings may indirectly help in combatting the spread of opioid addiction.

Hydrogel-based oral wound dressings are known in the art and sold commercially for the topical treatment of wounds in the oral cavity. Some oral hydrogels, such as Afta-Block™ Oral Gel (Alma Srl, Milan, Italy) and Aminogam® Gel (HANSAmed Limited, Ontario, Canada, incorporate hyaluronic acid or a salt thereof as a moisturizer and/or wound healing accelerator. Another hydrogel-based oral wound dressing, sold under the trademark SockIt! Gel™ (MCMP Co., Grand Prairie, Texas), incorporates beta-linked acylated mannans (Acemannan) and pectin as bioadhesive gelling agents in combination with xylitol and certain essential oils, and has been shown to be safe and effective in both alleviating pain and promoting wound healing.

There remains a need for new oral hydrogel compositions that are safe and effective in treating wounds in the oral cavity, and which have improved properties, e.g., stability, shelf life, and microbial resistance. The present invention provides such compositions and methods of using them for the treatment of wounds, e.g., sores, lesions, and ulcers, in the oral cavity. These and other advantages of the invention, as well as additional inventive features, will be apparent from the description of the invention provided herein.

### BRIEF SUMMARY OF THE INVENTION

The present invention provides a therapeutic composition in the form of a hydrogel which includes one or more polysaccharides, one or more polyols, one or more essential oils, and water, wherein the hydrogel has a pH of from about 6.2 to about 8.5.

In some embodiments, the hydrogel of the invention includes from about 0.1 wt. % to about 5 wt. % of one or more polysaccharides, from about 0.1 wt. % to about 5 wt. % of one or more polyols, from about 0.05 wt. % to about 0.5 wt. % of one or more essential oils, from about 0.1 wt. % to about 2 wt. % of a bicarbonate salt, and from about 90 wt. % to about 95 wt. % water, wherein the hydrogel has a pH of about 6.2 to about 8.5.

For example, the hydrogel of the invention may include, e.g., from about 0.1 wt. % to about 5 wt. % of guar gum, from about 0.1 wt. % to about 5 wt. % of acemannan, from about 0.1 wt. % to about 5 wt. % of xylitol, from about 0.01 wt. % to about 0.5 wt. % of cinnamon oil, from about 0.02 wt. % to about 0.5 wt. % of clove oil, from about 0.01 wt. % to about 0.5 wt. % of thyme oil, from about 0.1 wt. % to about 1 wt. % of sodium bicarbonate, from about 0.01 wt. % to about 0.5 wt. % of one or more optional preservatives, and from about 90 wt. % to about 95 wt. % water, wherein the hydrogel has a pH of from about 6.2 to about 8.5, e.g., from about 7.5 to about 8.5, e.g., from about 8.0 to about 8.5.

The hydrogel of the invention also may include, e.g., from about 0.1 wt. % to about 5 wt. % of pectin, from about 0.1 wt. % to about 5 wt. % of acemannan, from about 0.1 wt. % to about 5 wt. % of xylitol, from about 0.01 to about 0.5 wt. % of cinnamon oil, from about 0.02 wt. % to about 0.5 wt. % of clove oil, from about 0.1 wt. % to about 0.5 wt. % of thyme oil, from about 0.1 wt. % to about 1 wt. % of sodium bicarbonate, and from about 90 wt. % to about 95 wt. % water, wherein the hydrogel has a pH of from about 6.2 to about 8.5, e.g., from about 7.2 to about 7.8.

The present invention further provides a method of treating a wound in the oral cavity, e.g., sores, injuries, lesions, and ulcers of the oral mucosa, by topically applying an effective amount of the hydrogel of the invention to the wound in the oral cavity. An effective amount may include, e.g., an amount of hydrogel needed to form a barrier on the surface of the wound that is sufficiently protective to alleviate pain and/or promote wound healing.

### BRIEF DESCRIPTION OF THE DRAWINGS

The invention is best understood from the following detailed description when read in conjunction with the accompanying drawings. According to common practice, the dimensions and other various features of the drawings are not necessarily to-scale, and may be arbitrarily expanded or reduced for clarity.

FIG. 1 depicts a photograph of an exposed tissue sample taken before application of a hydrogel of the invention.

FIG. 2 depicts a photograph of an exposed tissue sample taken immediately after application of a hydrogel of the invention.

FIG. 3 depicts a photograph of an exposed tissue sample taken 1 hour after application of a hydrogel of the invention.

FIG. 4 depicts a photograph of an exposed tissue sample taken 2 hours after application of a hydrogel of the invention.

FIG. 5 depicts a photograph of an exposed tissue sample taken 4 hours after application of a hydrogel of the invention.

FIG. 6 depicts a photograph of an exposed tissue sample taken 24 hours after application of a hydrogel of the invention.

FIG. 7 depicts a graph comparing changes in viscosity over time exhibited by a hydrogel of the invention relative to a known hydrogel.

FIG. 8 depicts a graph comparing changes in pH over time exhibited by a hydrogel of the invention relative to a known hydrogel.

FIG. 9 depicts a graph comparing changes in viscosity over time exhibited by a hydrogel of the invention relative to a known hydrogel.

### DETAILED DESCRIPTION OF THE INVENTION

The invention provides a therapeutic composition in the form of a hydrogel which includes one or more polysaccharides (e.g., from about 0.1 to about 5 wt. % of one or more polysaccharides), one or more polyols (e.g., from about 0.1 to about 5 wt. % of one or more polyols), one or more essential oils (e.g., from about 0.05 to about 0.5 wt. % of one or more essential oi), one or more optional preservatives or antimicrobial agents, and water (e.g., from about 90 to about 95 wt. % water), wherein the hydrogel has a pH of from about 6.2 to about 8.5.

The one or more polysaccharides may include any suitable naturally occurring or synthetic polysaccharide, preferably a polysaccharide with bioadhesive, e.g., mucoadhesive, properties, and more preferably a bioadhesive polysaccharide that can form and/or can be incorporated into a hydrogel. Examples of polysaccharides include, e.g., guar gum, acemannan, pectin, celluloses, cellulose derivatives, e.g., carboxymethylcellulose, locust bean flour, starches, e.g., waxy maize starch, alginate, guar flour, carrageenan, karaya gum, gellan gum, konjac glucomannan, galactomannan, tara stone flour, propylene glycol alginate, sodium hyaluronate, tragacanth, tara gum, welan gum, agar, xanthan gum, and the like, and combinations thereof. In certain embodiments, the one or more polysaccharides includes one or more of guar gum, acemannan, pectin, carrageenan, karaya gum, gellan gum, konjac glucomannan, galactomannan, tara stone flour, propylene glycol alginate, sodium hyaluronate, tragacanth, tara gum, welan gum, agar, and xanthan gum. In some embodiments, the one or more polysaccharides includes, e.g., acemannan, guar gum, pectin, or a combination thereof. For instance, in some embodiments the one or more polysaccharides may include a combination of acemannan and guar gum, or a combination of acemannan and pectin.

The hydrogel of the invention may include any suitable amount of the one or more polysaccharides. In some embodiments, the hydrogel of the invention includes from about 0.1 wt. % to about 5 wt. % of the one or more polysaccharides. For example, the hydrogel of the invention may include from about 0.5 wt. % to about 5 wt. %, e.g., from about 1 wt. % to about 5 wt. %, e.g., from about 2 wt. % to about 5 wt. %, e.g., from about 2 wt. % to about 4 wt. %, e.g., from about 2 wt. % to about 3 wt. %, or, e.g., from about 3 wt. % to about 5 wt. % of the one or more polysaccharides. In one embodiment, the one or more polysaccharides includes, e.g., from about 0.1 wt. % to about 5 wt. % of guar gum and from about 0.1 wt. % to about 5 wt. % of acemannan, e.g., from about 1 wt. % to about 3 wt. % guar gum and from about 1 wt. % to about 3 wt. % acemannan, e.g., from about 1 wt. % to about 2 wt. % guar gum and from about 1 wt. % to about 2 wt. % acemannan, e.g., from about 1 wt. % to about 1.5 wt. % guar gum and from about 1 wt. % to about 1.5 wt. % acemannan, e.g., about 1.3 wt. % guar gum and about 1.3 wt. % acemannan. In another embodiment, the one or more polysaccharides includes, e.g., from about 0.1 wt. % to about 5 wt. % of pectin and from about 0.1 wt. % to about 5 wt. % of acemannan, e.g., from about 2 wt. % to about 3 wt. % pectin and from about 1 wt. % to about 1.5 wt. % acemannan, e.g., about 2.5 wt. % pectin and about 1.3 wt. % acemannan.

The one or more polyols may include any suitable naturally occurring or synthetic polyol (e.g., sugar alcohol). For instance, the polyol may include 4 carbons or more, e.g., 5 carbons or more, or e.g., 6 carbons or more, and may include, e.g., 4 or more hydroxyl groups, e.g., 5 or more hydroxyl groups, or e.g., 6 or more hydroxyl groups. In some embodiments, the polyol includes one hydroxyl group for each carbon atom in the chain that defines the alcohol, e.g., a polyol with the same number of hydroxyl groups as carbon atoms in the chain that defines the alcohol. Examples of one or more polyols may include xylitol, threitol, arabitol, ribitol, mannitol, sorbitol, galactitol, fucitol, iditol, inositol, and the like, and combinations thereof. In some embodiments, the one or more polyols includes xylitol, erythritol, threitol, mannitol, sorbitol, or a combination thereof. In a preferred embodiment, the one or more or polyols is xylitol.

The hydrogel of the invention may include, e.g., from about 0.1 wt. % to about 5 wt. % of the one or more polyols. For example, the hydrogel of the invention may include from about 0.5 wt. % to about 5 wt. % of one or more polyols, e.g., from about 1 wt. % to about 5 wt. % of one or more polyols, e.g., from about 1 wt. % to about 4 wt. % of one or more polyols, e.g., from about 1 to about 3 wt. % of one or more polyols, or e.g., from about 1.5 wt. % to about 2 wt. % of one or more polyols. In some embodiments, the polyol includes, e.g., from about 0.5 wt. % to about 5 wt. % xylitol, e.g., from about 1 wt. % to about 5 wt. % xylitol, e.g., from about 1 wt. % to about 4 wt. % xylitol, e.g., from about 1 wt. % to about 3 wt. % xylitol, e.g., from about 1 wt. % to about 2.5 wt. % xylitol, e.g., from about 1 wt. % to about 2 wt. % xylitol, e.g., from about 1.5 wt. % to about 2 wt. % xylitol, e.g., from about 1.5 wt. % to about 2.5 wt. % xylitol, e.g., about 1.9 wt. % xylitol.

The hydrogel of the invention preferably includes one or more essential oils. The one or more essential oils may include any suitable naturally occurring essential oil (e.g., volatile oil, ethereal oil, or aetherolea). The essential oil can be obtained by any suitable method (e.g., extraction, distillation, or the like) from any suitable plant. An exemplary list of essential oils includes, but is not limited to, cinnamon oil, clove oil, thyme oil, anise oil, bergamot oil, eucalyptus oil, ginger oil, lavender oil, peppermint oil, rosemary oil, spearmint oil, wintergreen oil, and the like, and combinations thereof. In some embodiments, the essential oil includes cinnamon oil, clove oil, thyme oil, or a combination thereof. In one embodiment, the one or more essential oils includes a combination of cinnamon oil, clove oil, and thyme oil.

The hydrogel of the invention may include any suitable amount of the one or more essential oils (e.g., volatile oils, ethereal oils, or aetheroleas). In some embodiments, the hydrogel of the invention includes, e.g., from about 0.05 wt. % to about 0.5 wt. % of one or more essential oils, e.g., from about 0.05 to about 0.4 wt. % of one or more essential oils, e.g., from about 0.05 wt. % to about 0.3 wt. % of one or more essential oils, e.g., from about 0.1 wt. % to about 0.5 wt. % of one or more essential oils, e.g., from about 0.1 wt. % to about 0.4 wt. % of one or more essential oils, e.g., from about 0.1 wt. % to about 0.3 wt. % of one or more essential oils. In some embodiments, the hydrogel of the invention includes from about 0.01 wt. % to about 0.1 wt. % cinnamon oil, from about 0.01 wt. % to about 0.1 wt. % clove oil, and from about 0.01 wt. % to about 0.1 wt. % thyme oil. In one embodiment, the hydrogel of the invention includes, e.g., about 0.05 wt. % cinnamon oil, about 0.07 wt. % clove oil, and about 0.03 wt. % thyme oil.

The hydrogel of the invention may include any amount of water suitable for hydrogel formation. In some embodiments, the hydrogel of the invention may include, e.g., from about 90 wt. % to about 95 wt. % water, e.g., from about 91 wt. % to about 95 wt. % water, e.g., from about 92 to about 95 wt. % water, e.g., from about 93 wt. % to about 95 wt. % water, e.g., from about 93 wt. % to about 94 wt. % water (e.g., about 94 wt. % water), e.g., from about 94 wt. % to about 95 wt. % water (e.g., about 95 wt. % water).

In some embodiments, the hydrogel of the invention may further include a buffering agent which may include, for example, a bicarbonate salt. The bicarbonate salt may include any suitable compound that includes a bicarbonate or ⁻HCO₃ ion/counter ion. The bicarbonate salts may include, without limitation, e.g., lithium bicarbonate, sodium bicarbonate, and potassium bicarbonate. In some embodiments, the bicarbonate salt is potassium bicarbonate or sodium bicarbonate, and is preferably sodium bicarbonate.

The hydrogel of the invention may include any suitable amount of bicarbonate salt. In some embodiments, the hydrogel of the invention may include, e.g., from about 0.1 wt. % to about 2 wt. % of the bicarbonate salt, e.g., sodium bicarbonate. For example, the hydrogel of the invention may include from about 0.1 wt. % to about 1 wt. %, e.g., from about 0.1 wt. % to about 0.5 wt. %, e.g., from about 0.2 wt. % to about 2 wt. %, e.g., from about 0.2 wt. % to about 1 wt. %, e.g., from about 0.2 wt. % to about 0.6 wt. %, e.g., from about 0.2 wt. % to about 0.5 wt. %, or e.g., from about 0.3 wt. % to about 0.5 wt. % of the bicarbonate salt, e.g., sodium bicarbonate.

The hydrogel of the invention also may include one or more optional preservatives and/or antimicrobial agents such as, for example, sorbic acid or a salt thereof (e.g., sodium sorbate, potassium sorbate), benzoic acid or a salt thereof (e.g., sodium benzoate, potassium benzoate), one or more sulfites (e.g., sodium sulfite) and/or bisulfites (e.g., sodium metabisulfite), one or more nitrites (e.g., sodium nitrite), ascorbic acid or a salt thereof, alpha tocopherol or a derivative thereof, and the like, and combinations thereof. In some embodiments, the hydrogel of the invention optionally includes a sorbate salt such as, e.g., potassium sorbate. The hydrogel of the invention may include any suitable amount of optional preservative(s)/antimicrobial agent(s). For example, the hydrogel of the invention may include from about 0.01 wt. % to about 0.5 wt. % of one or more optional preservative(s)/antimicrobial agent(s), e.g., from about 0.05 wt. % to about 0.5 wt. % of one or more optional preservative(s)/antimicrobial agent(s), e.g., from about 0.1 wt. % to about 0.5 wt. % of one or more optional preservative(s)/antimicrobial agent(s), e.g., from about 0.1 wt. % to about 0.4 wt. % of one or more optional preservative(s)/antimicrobial agent(s), e.g., from about 0.1 wt. % to about 0.3 wt. % of one or more optional preservative(s)/antimicrobial agent(s). In some embodiments, the hydrogel of the invention may optionally include, e.g., about 0.2 wt. % of potassium sorbate.

The hydrogel of the invention preferably has a pH of from about 6.2 to about 8.5. For example, the hydrogel of the invention may have a pH of from about 7.0 to about 8.5, e.g., from about 7.5 to about 8.5, e.g., from about 7.6 to about 8.5, e.g., e.g., from about 7.7 to about 8.5, e.g., from about 7.8 to about 8.5, e.g., from about 7.9 to about 8.5, from about 8.0 to about 8.5. In some embodiments, the hydrogel of the invention has a pH of from about 8.0 to about 8.5, e.g., from about 8.1 to about 8.5, e.g., from about 8.2 to about 8.5, e.g., from about 8.3 to about 8.5, e.g., from about 8.4 to about 8.5, e.g., from about 8.0 to about 8.5, e.g., from about 8.0 to about 8.4, e.g., from about 8.0 to about 8.3, e.g., from about 8.0 to about 8.2, e.g., from about 8.0 to about 8.1, e.g., from about 8.1 to about 8.5, e.g., from about 8.1 to about 8.4, e.g., from about 8.1 to about 8.3, e.g., from about 8.1 to about 8.2, e.g., from about 8.2 to about 8.5, e.g., from about 8.2 to about 8.4, e.g., from about 8.2 to about 8.3, e.g., about 8.2 or about 8.3, when measured at ambient temperature and pressure. In other embodiments, the hydrogel of the invention may have a pH of, e.g., from about 7.2 to about 7.8, e.g., from about 7.3 to about 7.8, e.g., from about 7.4 to about 7.8, e.g., from about 7.5 to about 7.8, e.g., from about 7.6 to about 7.8, e.g., from about 7.6 to about 7.7, e.g., from about 7.5 to about 7.7, e.g., from about 7.5 to about 7.6, e.g., about 7.6, when measured at ambient temperature and pressure.

In some embodiments, the hydrogel of the invention may include, e.g., from about 0.1 wt. % to about 5 wt. % of one or more polysaccharides, from about 0.1 wt. % to about 5 wt. % of one or more polyols, from about 0.05 wt. % to about 0.5 wt. % of one or more essential oils, from about 0.1 wt. % to about 2 wt. % of a bicarbonate salt, from about 0.01 wt. % to about 0.5 wt. % of one or more optional preservative(s)/antimicrobial agent(s), and from about 90 wt. % to about 95 wt. % water, wherein the hydrogel has a pH of from about 7.0 to about 8.5, e.g., from about 7.5 to about 8.5, e.g., from about 8.0 to about 8.5, e.g., from about 8.1 to about 8.5, e.g., from about 8.1 to about 8.4, e.g., from about 8.1 to about 8.3, e.g., from about 8.1 to about 8.2, e.g., from about 8.2 to about 8.5, e.g., from about 8.2 to about 8.4, e.g., from about 8.2 to about 8.3, e.g., about 8.2 or about 8.3. In other embodiments, the hydrogel of the invention may include from about 0.1 wt. % to about 5 wt. % of one or more polysaccharides, from about 0.1 wt. % to about 5 wt. % of one or more polyols, from about 0.05 wt. % to about 0.5 wt. % of one or more essential oils, from about 0.1 wt. % to about 2 wt. % of a bicarbonate salt, and from about 90 wt. % to about 95 wt. % water, wherein the hydrogel has a pH of from about 7.2 to about 7.8, e.g., from about 7.5 to about 7.7, e.g., about 7.6.

In some embodiments, the hydrogel of the invention may include, e.g., from about 0.1 to about 5 wt. % of guar gum, from about 0.1 to about 5 wt. % of acemannan, from about 0.1 to about 5 wt. % of xylitol, from about 0.01 to about 0.1 wt. % of cinnamon oil, from about 0.01 to about 0.1 wt. % of clove oil, from about 0.01 to about 0.1 wt. % of thyme oil, from about 0.1 to about 1 wt. % sodium bicarbonate, from about 0.01 wt. % to about 0.5 wt. % of one or more optional preservatives and/or antimicrobial agents, and from about 90 to about 95 wt. % water, wherein the hydrogel has a pH of from about 7.0 to about 8.5, e.g., from about 7.5 to about 8.5, e.g., from about 8.0 to about 8.5, e.g., a pH of from about 8.1 to about 8.5, e.g., from about 8.1 to about 8.4, e.g., from about 8.1 to about 8.3, e.g., from about 8.1 to about 8.2, e.g., from about 8.2 to about 8.5, e.g., from about 8.2 to about 8.4, e.g., from about 8.2 to about 8.3, e.g., about 8.2 or about 8.3. In other embodiments, the hydrogel of the invention may include, e.g., from about 0.1 to about 5 wt. % of pectin, from about 0.1 to about 5 wt. % of acemannan, from about 0.1 to about 5 wt. % of xylitol, from about 0.01 to about 0.1 wt. % of cinnamon oil, from about 0.01 to about 0.1 wt. % of clove oil, from about 0.01 to about 0.1 wt. % of thyme oil, from about 0.1 to about 1 wt. % sodium bicarbonate, and from about 90 to about 95 wt. % water, wherein the hydrogel has a pH of from about 7.2 to about 7.8, e.g., from about 7.5 to about 7.7, e.g., about 7.6.

Preferably, one or more of the ingredients/components in the hydrogel of the invention is food grade or of similar quality/suitability, and most preferably all ingredients/components in the hydrogel of the invention are food grade or of similar quality/suitability.

The hydrogel of the invention may have any suitable viscosity. For example, the hydrogel of the invention may have a viscosity that is sufficiently high for maintaining hydrogel structure during storage while allowing the hydrogel to be dispensed easily through the opening of a syringe applicator of the type commonly used for dispensing oral hydrogels. In some embodiments, the hydrogel of the invention has a viscosity, e.g., of from about 10,000 cP to about 35,000 cP, e.g., from 10,000 cP to about 35,000 cP, e.g., from about 10,000 cP to about 30,000 cP, e.g., from about 10,000 cP to about 25,000 cP, e.g., from about 15,000 cP to about 35,000 cP, e.g., from about 15,000 cP to about 30,000 cP, e.g., from about 15,000 cP to about 25,000 cP, e.g., from about 20,000 cP to about 25,000 cP, at about 25° C. as measured by a calibrated rotary viscometer with a rotary speed of 12 RPM and a rotor size of 4.

The hydrogel of the invention surprisingly and unexpectedly has been found to exhibit improved physical stability relative to comparable hydrogels as demonstrated by its ability to resist changes or decline/reduction in viscosity, and to resist phase separation (of oil components), over time, e.g., over long-term storage, relative to comparable hydrogels. The hydrogel of the invention preferably exhibits less than about 50% reduction in viscosity, e.g., less than about 40% reduction in viscosity, e.g., less than about 30% reduction in viscosity, e.g., less than about 20% reduction in viscosity, after up to 24 months, e.g., after 12-24 months, of storage at about 25° C., e.g., 25±2° C., as measured by a calibrated rotary viscometer with a rotary speed of 12 RPM and a rotor size of 4. The hydrogel of the invention more preferably exhibits less than about 50% reduction in viscosity, e.g., less than about 40% reduction in viscosity, e.g., less than about 30% reduction in viscosity, e.g., less than about 20% reduction in viscosity, after up to 24 months, e.g., after 12-24 months, of storage at about 25° C., e.g., 25±2° C., as measured by a calibrated rotary viscometer with a rotary speed of 12 RPM and a rotor size of 4. In some embodiments, the hydrogel of the invention exhibits less than about 30% reduction in viscosity, e.g., less than about 20% reduction in viscosity, after at least about 12 months, e.g., after up to 24 months, e.g., after 12-24 months, of storage at about 25° C., e.g., 25±2° C., as measured by a calibrated rotary viscometer with a rotary speed of 12 RPM and a rotor size of 4.

The hydrogel of the invention also surprisingly and unexpectedly has been found to exhibit improved chemical stability as demonstrated by improved pH stability, e.g., resistance to change in pH, over time, e.g., over long-term, storage relative to comparable hydrogels. The hydrogel of the invention preferably exhibits a change of, e.g., no more than about 0.5 pH units, e.g., no more than about 0.4 pH units, e.g., no more than about 0.3 pH units, e.g., no more than about 0.2 pH units, e.g., no more than about 0.1 pH units, after up to 24 months, e.g., after 12-24 months, of storage at about 25° C., e.g., 25±2° C. In some embodiments, the hydrogel of the invention exhibits a pH drop of, e.g., no more than about 0.5 pH units, e.g., no more than about 0.4 pH units, e.g., no more than about 0.3 pH units, e.g., no more than about 0.2 pH units, e.g., no more than about 0.1 pH units, after up to 24 months, e.g., after 12-24 months, of storage at about 25° C., e.g., 25±2° C.

The hydrogel of the invention also surprisingly and unexpectedly has been found to exhibit excellent resistance to microbial contamination. The hydrogel of the invention may exhibit a total aerobic microbial count of, e.g., less than about 100 CFU/g after storage at 25±2° C. for up to 24 months, e.g., less than about 50 CFU/g after storage at 25±2° C. for up to 24 months, e.g., less than about 10 CFU/g after storage at 25±2° C. for up to 24 months, e.g., less than about 5 CFU/g after storage at 25±2° C. for up to 24 months, e.g., less than about 2 CFU/g after storage at 25±2° C. for up to 24 months, and even, e.g., less than about 1 CFU/g after storage at 25±2° C. for up to 24 months. The hydrogel of the invention may exhibit a total yeast and mold count of, e.g., less than about 10 CFU/g after storage at 25±2° C. for up to 24 months, e.g., less than about 5 CFU/g after storage at 25±2° C. for up to 24 months, e.g., less than about 4 CFU/g after storage at 25±2° C. for up to 24 months, e.g., less than about 3 CFU/g after storage at 25±2° C. for up to 24 months, and even, e.g., less than about 2 CFU/g after storage at 25±2° C. for up to 24 months.

The present invention further provides a method of treating a wound in the oral cavity, e.g., sores, injuries, lesions, and ulcers of the oral mucosa, by topically applying an effective amount of the hydrogel of the invention to the wound in the oral cavity. An effective amount includes an amount of hydrogel effective to form a barrier on the surface of the wound that is sufficiently protective to alleviate pain and/or promote wound healing. The hydrogel of the invention adheres to the oral tissue and forms a protective barrier between the wound and further irritation or contamination. The hydrogel of the invention is effective in alleviating pain associated with oral wounds, e.g., sores, injuries, lesions, or ulcers of the oral mucosa. The hydrogel of the invention also is effective in accelerating wound healing. The hydrogel of the invention further provides a moist wound environment needed to promote optimal wound healing. Without wishing to be bound by any particular theory, the hydrogel of the invention is believed to alleviate pain and/or accelerate healing, at least in part, by adhering to the wound surface, and/or protecting the wound from contamination and irritation by forming a protective barrier that conforms to the contours of the wound and mimics the protective function of natural mucosa.

The hydrogel of the invention may be applied to a wound in the oral cavity using any suitable method. For example, the hydrogel of the invention may be applied directly to the wound surface with a syringe applicator or by other means, e.g., manually.

### EMBODIMENTS

(1) In embodiment (1) is presented a therapeutic composition in the form of a hydrogel, suitable for treating oral wounds, the hydrogel comprising one or more polysaccharides, one or more polyols, one or more essential oils, and water, wherein the hydrogel has a pH of from about 6.2 to about 8.5.

(2) In embodiment (2) is presented the composition of embodiment (1), wherein the hydrogel has a pH of from about 7.0 to about 8.5.

(3) In embodiment (3) is presented the composition of embodiment (1) or (2), wherein the hydrogel has a pH of from about 8.0 to about 8.5, or from about 7.0 to about 8.0.

(4) In embodiment (4) is presented the composition of any one of embodiments (1)-(3), wherein the hydrogel further comprises a bicarbonate salt.

(5) In embodiment (5) is presented the composition of embodiment (4), wherein the bicarbonate salt is lithium bicarbonate, sodium bicarbonate, potassium bicarbonate, or a combination thereof.

(6) In embodiment (6) is presented the composition of embodiment (4) or (5), wherein the hydrogel comprises from about 0.1 wt. % to about 2 wt. % of the bicarbonate salt.

(7) In embodiment (7) is presented the composition of any one of embodiments (4)-(6), wherein the hydrogel comprises from about 0.1 wt. % to about 1 wt. % of the bicarbonate salt.

(8) In embodiment (8) is presented the composition of any one of embodiments (4)-(7), wherein the hydrogel comprises from about 0.2 wt. % to about 0.6 wt. % of the bicarbonate salt.

(9) In embodiment (9) is presented the composition of any one of embodiments (1)-(8), wherein the one or more polysaccharides comprises acemannan, guar gum, pectin, or a combination thereof.

(10) In embodiment (10) is presented the composition of any one of embodiments (1)-(9), wherein the hydrogel comprises from about 0.1 wt. % to about 5 wt. % of the one or more polysaccharides.

(11) In embodiment (11) is presented the composition of any one of embodiments (1)-(10), wherein the hydrogel comprises from about 1 wt. % to about 5 wt. % of the one or more polysaccharides.

(12) In embodiment (12) is presented the composition of any one of embodiments (1)-(11), wherein the hydrogel comprises from about 2 wt. % to about 5 wt. % of the one or more polysaccharides.

(13) In embodiment (13) is presented the composition of any one of embodiments (1)-(12), wherein the one or more polyols includes xylitol, erythritol, threitol, mannitol, sorbitol, or a combination thereof.

(14) In embodiment (14) is presented the composition of any one of embodiments (1)-(13), wherein the hydrogel comprises from about 0.1 wt. % to about 5 wt. % of the one or more polyols.

(15) In embodiment (15) is presented the composition of any one of embodiments (1)-(14), wherein the hydrogel comprises from about 1 wt. % to about 5 wt. % of the one or more polyols.

(16) In embodiment (16) is presented the composition of any one of embodiments (1)-(15), wherein the hydrogel comprises from about 1 wt. % to about 3 wt. % of the one or more polyols.

(17) In embodiment (17) is presented the composition of any one of embodiments (1)-(15), wherein the one or more essential oils includes cinnamon, clove, thyme, anise, bergamot, eucalyptus, ginger, lavender, peppermint, rosemary, spearmint, wintergreen, or a combination thereof.

(18) In embodiment (18) is presented the composition of any one of embodiments (1)-(17), wherein the hydrogel comprises from about 0.05 wt. % to about 0.5 wt. % of the one or more essential oils.

(19) In embodiment (19) is presented the composition of any one of embodiments (1)-(18), wherein the hydrogel comprises from about 0.05 wt. % to about 0.4 wt. % of the one or more essential oils.

(20) In embodiment (20) is presented the composition of any one of embodiments (1)-(19), wherein the hydrogel comprises from about 0.05 wt. % to about 0.3 wt. % of the one or more essential oils.

(21) In embodiment (21) is presented the composition of any one of embodiments (1)-(20), wherein the hydrogel comprises from about 90 wt. % to about 95 wt. % water.

(22) In embodiment (22) is presented the composition of any one of embodiments (1)-(21), wherein the hydrogel comprises from about 92 wt. % to about 95 wt. % water.

(23) In embodiment (23) is presented the composition of any one of embodiments (1)-(22), wherein the hydrogel exhibits less than about 30% reduction in viscosity after up to 24 months of storage at 25° C.

(24) In embodiment (24) is presented the composition of any one of embodiments (1)-(23), wherein the hydrogel has a total aerobic microbial count of less than 5 CFU/g after storage at 25±2° C. for up to 24 months.

(25) In embodiment (25) is presented the composition of any one of embodiments (1)-(24), wherein the hydrogel has a total aerobic microbial count of less than 2 CFU/g after storage at 25±2° C. for up to 24 months.

(26) In embodiment (26) is presented the composition of any one of embodiments (1)-(25), wherein the hydrogel has a total aerobic microbial count of less than 1 CFU/g after storage at 25±2° C. for up to 24 months.

(27) In embodiment (27) is presented the composition of any one of embodiments (1)-(26), wherein the hydrogel has a total yeast and mold count of less than 5 CFU/g after storage at 25±2° C. for up to 24 months.

(28) In embodiment (28) is presented the composition of any one of embodiments (1)-(27), wherein the hydrogel has a total yeast and mold count of less than 2 CFU/g after storage at 25±2° C. for up to 24 months.

(29) In embodiment (29) is presented the composition of any one of embodiments (1)-(28), wherein the hydrogel consists essentially of from about 0.1 wt. % to about 5 wt. % of one or more polysaccharides, from about 0.1 wt. % to about 5 wt. % of one or more polyols, from about 0.05 wt. % to about 0.5 wt. % of one or more essential oils, from about 0.1 wt. % to about 2 wt. % of a bicarbonate salt, optionally from about 0.01 wt. % to about 0.5 wt. % of one or more preservatives and/or antimicrobial agents, and from about 90 wt. % to about 95 wt. % water, wherein the therapeutic composition has a pH of from about 6.2 to about 8.5.

(30) In embodiment (30) is presented the composition of any one of embodiments (1)-(29), wherein the hydrogel comprises or consists of from about 1 wt. % to about 1.5 wt. % guar gum, from about 1 wt. % to about 1.5 wt. % acemannan, from about 1.5 wt. % to about 2 wt. % xylitol, from about 0.01 wt. % to about 0.1 wt. % of cinnamon oil, from about 0.01 to about 0.1 wt. % of clove oil, from about 0.01 to about 0.1 wt. % of thyme oil, from about 0.1 to about 1 wt. % of sodium bicarbonate, optionally from about 0.01 wt. % to about 0.5 wt. % of one or more preservatives and/or antimicrobial agents, and from about 90 to about 95 wt. % water, and wherein the hydrogel has a pH of from about 6.2 to about 8.5, e.g., from about 7.0 to about 8.5, e.g., from about 7.5 to about 8.5, e.g., from about 8.0 to about 8.5.

(31) In embodiment (31) is presented the composition of any one of embodiments (1)-(30), wherein the hydrogel comprises or consists of about 1.3 wt. % guar gum, about 1.3 wt. % acemannan, about 1.9 wt. % xylitol, about 0.05 wt. % cinnamon oil, about 0.07 wt. % clove oil, about 0.03 wt. % thyme oil, about 0.4 wt. % sodium bicarbonate, optionally about 0.2 wt. % potassium sorbate, and about 94-95 wt. % water, wherein the hydrogel has a pH of from about 6.2 to about 8.5, e.g., from about 7.0 to about 8.5, e.g., from about 7.5 to about 8.5, e.g., from about 8.0 to about 8.5, e.g., about 8.2-8.3.

### EXAMPLES

The following examples further illustrate the invention but should not be construed as in any way limiting its scope.

### Example 1

This example describes a method for preparing an exemplary hydrogel of the invention. The components and concentrations of the hydrogel are described in Table 1.

TABLE 1
Inventive Composition

| Ingredient | % (w/w) | Function |
|---|---|---|
| Water | 93.749 | Solvent |
| Pectin | 2.5 | Bioadhesive Gelling Component |
| Acemannan | 1.3 | Bioadhesive Gelling Component |
| Xylitol | 1.9 | Sweetener |
| Sodium Bicarbonate | 0.4 | pH Buffer |
| Essential Oil of Cinnamon | 0.0454 | Therapeutic Agent, Preservative |
| Essential Oil of Clove | 0.0737 | Therapeutic Agent, Preservative |
| Essential Oil of Thyme | 0.0317 | Therapeutic Agent, Preservative |

Room-temperature water is agitated with an industrial immersion blender in a mixing apparatus, creating a vortex. Xylitol is poured in at a moderate rate and mixed until thoroughly dissolved. Once completely dissolved, sodium bicarbonate is added at a moderate rate and mixed until thoroughly dissolved. Next, Acemannan is added to the solution while mixing at a slow rate. The solution is mixed until all powders are dissolved. Pectin (BOC Sciences, Lot BS18ZX05161) is then added to the mixture at a slow rate to induce thickening and create a gel. The oils can be either pre-mixed or added one at a time to the thick solution. The gel is then mixed with the immersion blender for several minutes until completely uniform and free from lumps.

### Example 2

This example demonstrates the pH of an exemplary hydrogel of the invention, prepared according to Example 1, relative to a comparable commercial product, SockIt! Gel™.

The pH of the hydrogel of the invention and SockIt! Gel™ were measured using a calibrated pH meter. The pH meter was calibrated with Oakton Buffer Solutions having a pH of 7.00 and 4.01. The pH of the therapeutic composition of the invention and SockIt! Gel™ were measured in triplicate and the results are summarized in Table 2.

TABLE 2
pH Measurement

| | Inventive Composition | SockIt! Gel ™ |
|---|---|---|
| Trial 1 pH | 7.75 | 7.09 |
| Trial 2 pH | 7.53 | 7.09 |
| Trial 3 pH | 7.52 | 7.09 |
| Average pH | 7.6 | 7.09 |

The results summarized in Table 2 show that the hydrogel prepared according to Example 1 has a measurably higher pH than a comparable product, SockIt! Gel™

### Example 3

This example demonstrates the antimicrobial properties of a hydrogel of the invention relative to a comparable commercial product, SockIt! Gel™.

The total aerobic microbial count, total mold and yeast count, and screening for Staphylococcus aureus and Pseudomonas aeruginosa were performed using U.S. Pharmacopeia methodologies USP 42/NF 37, <61>, USP 42/NF 37, <61>, and USP 42/NF 37, <62>, respectively. The results are summarized in Table 3.

TABLE 3
Antimicrobial Properties

| | | Inventive | |
|---|---|---|---|
| Assay | Specification | Composition | SockIt! Gel ™ |
| Total Aerobic | <200 CFU/g | <1 CFU/g | 19 CFU/g |
| Microbial Count | | | |
| Total Molds and | <20 CFU/g | 1 CFU/g | 13 CFU/g |
| Yeasts Count | | | |
| Screening for | Absent/g | No Organisms | No Organisms |
| Staphylococcus aureus | | Detected | Detected |
| Screening for | Absent/g | No Organisms | No Organisms |
| Pseudomonas aeruginosa | | Detected | Detected |

The results summarized in Table 3 demonstrate that the hydrogel of the invention is more resistant to microbial contamination than a comparable hydrogel. The hydrogel of the invention exhibited a total aerobic microbial count, and total mold and yeast count, considerably below the maximum tolerable levels required by the specification. The results also demonstrate that the hydrogel of the invention, having less than one colony-forming unit (CFU) of total aerobic microbial count, and less than one CFU of total mold and yeast count, significantly outperformed SockIt! Gel™, which had 19 colony-forming units (CFUs) of aerobic microbes, and 13 CFUs of mold and yeast.

### Example 4

This example demonstrates the stability of a hydrogel of the invention relative to a comparable commercial product, SockIt! Gel™.

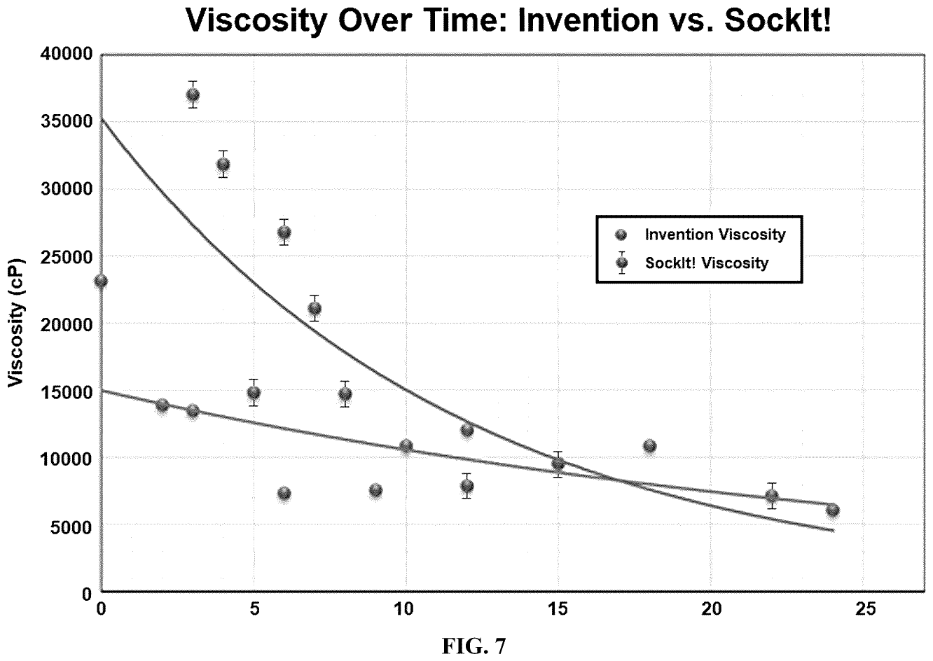

Viscosity is measured at 25° C. with a calibrated rotary viscometer having a rotor size of 4 and a rotary speed of 12 RPM. The viscosities of the hydrogel of the invention and SockIt! Gel™ (a comparable hydrogel) were determined periodically over 24 months. The results are graphically depicted in FIG. 7. The data demonstrate the hydrogel of the invention is considerably more stable than a comparable product as shown by its significantly greater resistance to reduction in viscosity over time relative to SockIt! Gel™.

### Example 5

This example demonstrates the effectiveness of a hydrogel of the invention in treating oral wounds.

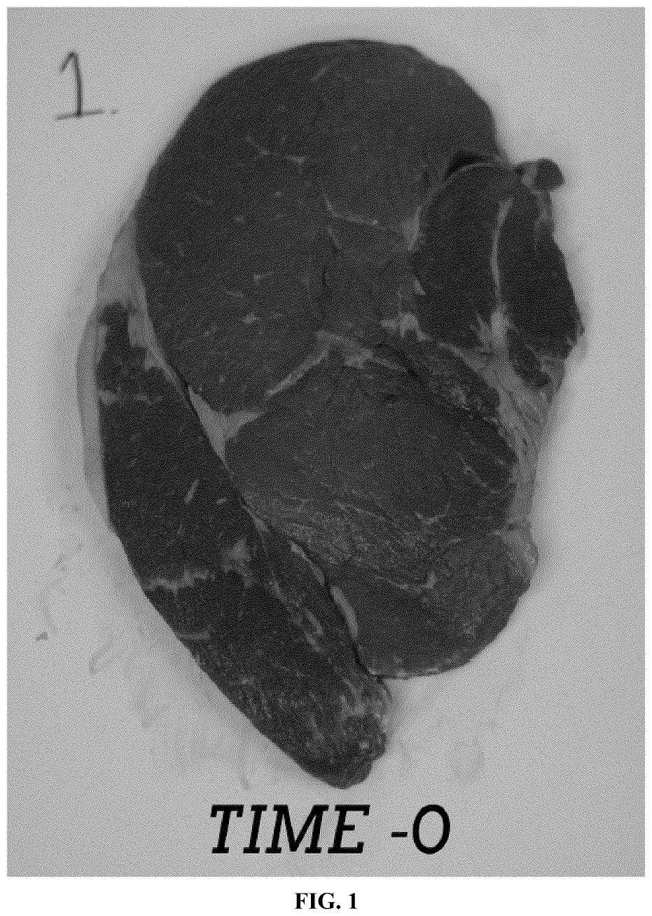

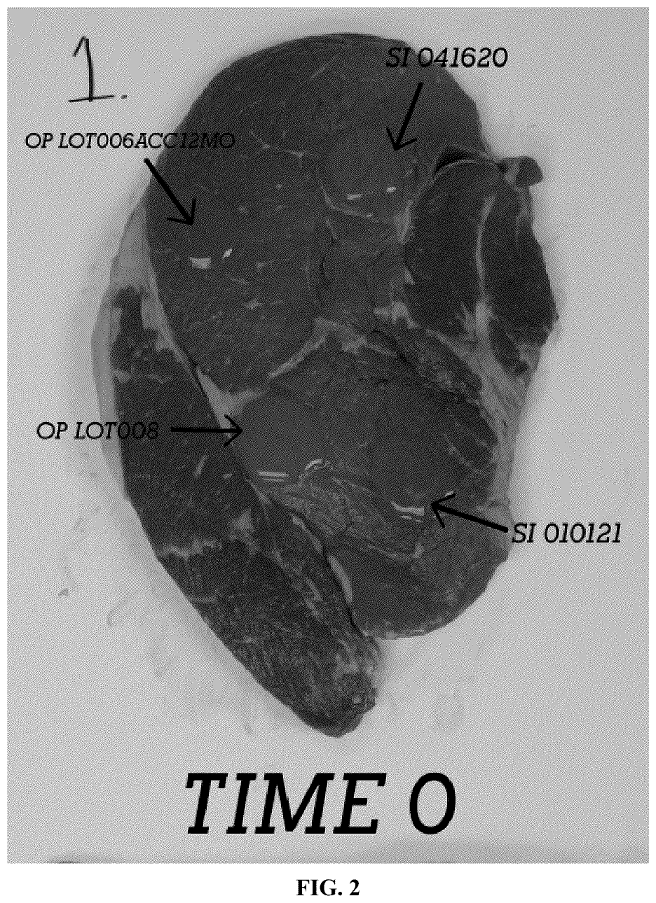

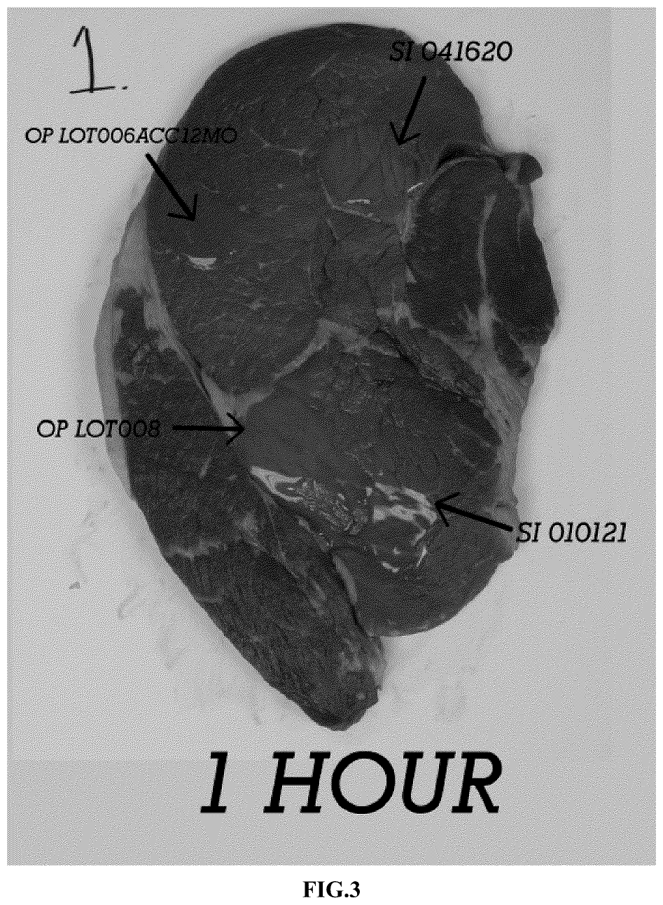

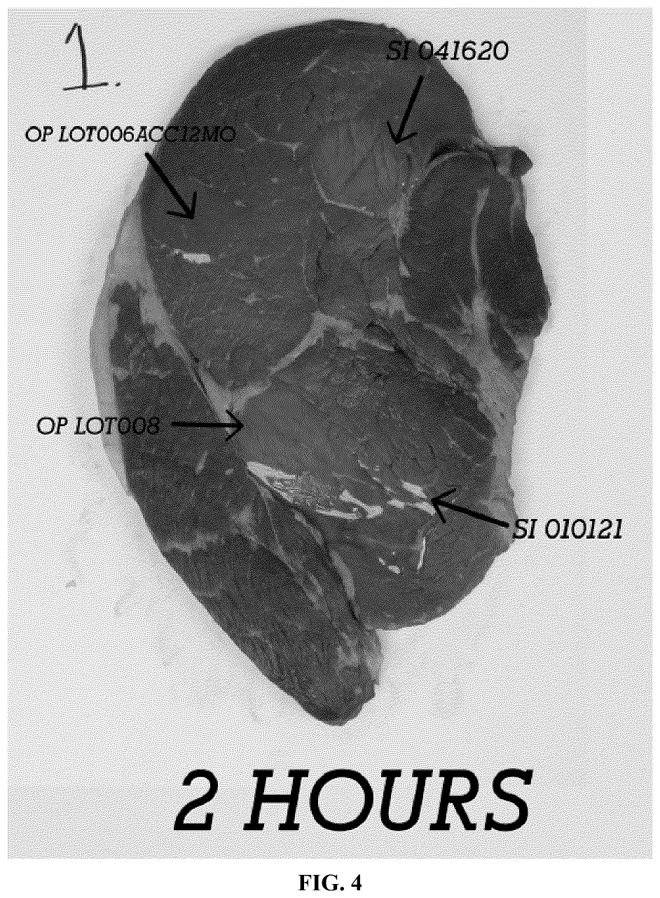

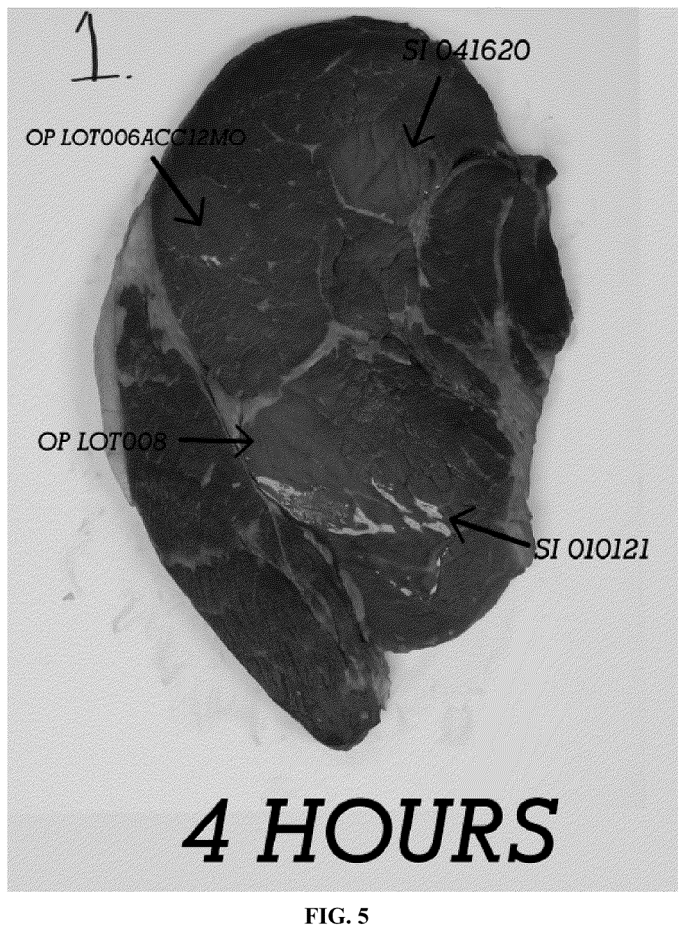

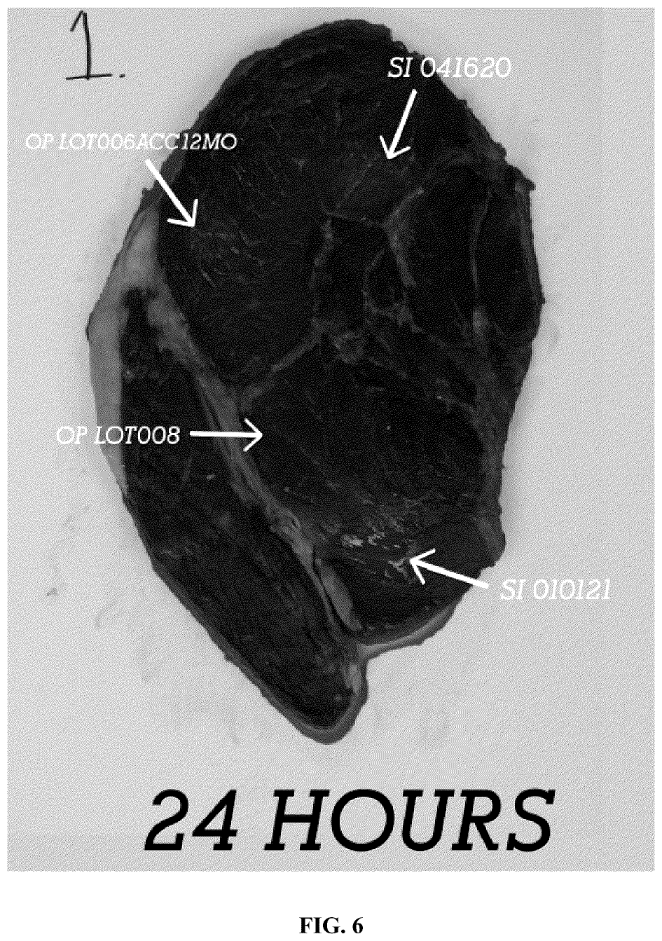

To a tissue sample (sirloin steak) with an exposed surface representative an oral wound was applied a hydrogel of the invention prepared according to Example 1 (Lot: 008), a hydrogel of the invention prepared according to Example 1 that has been aged at 37° C. for 12 months (Lot: 00612MO), SockIt! Gel™ (Lot: 041620), and SockIt! Gel™ (Lot: 010121). Photos of the tissue sample were taken before application (Time 0, FIG. 1)), immediately after application (Time 0, FIG. 2), 1 hour after application (FIG. 3), 2 hours after application (FIG. 4), 4 hours following application (FIG. 5), and 24 hours after application (FIG. 6).

As depicted in FIGS. 1-6, the hydrogel of the invention maintained a moist hydrogel barrier over the surface of the tissue sample for more than 24 hours, as evidenced by the slowing of the color change of the tissue sample. The color change in the tissue sample over time is believed to be due to the exposure of the tissue sample to oxygen. The hydrogel of the invention effectively provides a protective barrier that prevents oxygen from contacting the exposed tissue, thereby dramatically reducing oxidation relative to the unprotected surface. The ability of the hydrogel of the invention to inhibit discoloration of the tissue sample over time is demonstrative of its effectiveness in alleviating pain associated with wounds in the oral cavity, and in promoting the healing of wounds in the oral cavity.

### Example 6

This example describes an exemplary hydrogel of the invention which may be prepared according to Example 1, except using guar gum in place of pectin and obtaining a higher pH. The components and concentrations of the hydrogel are summarized in Table 4.

TABLE 4
Inventive Composition

| Ingredient | % (w/w) |
|---|---|
| Water | 94.9% |
| Xylitol | 1.9% |
| Guar Gum | 1.3% |
| Acemannan | 1.3% |
| Sodium Bicarbonate | 0.4% |
| Potassium Sorbate | 0.2% |
| Clove Oil | 0.0737% |
| Cinnamon Oil | 0.0454% |
| Thyme Oil | 0.0317% |

### Example 7

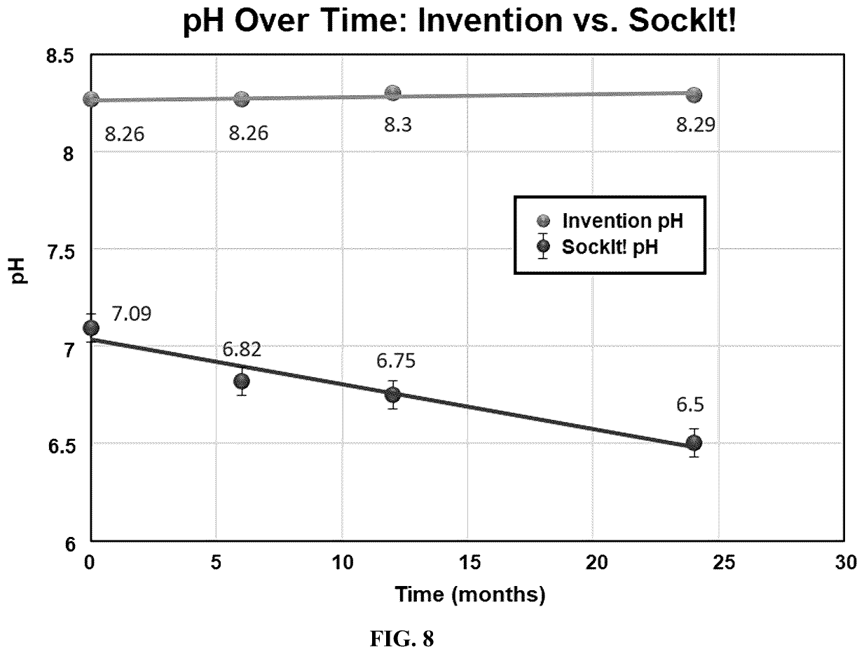

This example demonstrates the pH stability of the hydrogel of Example 6 relative to a comparable hydrogel, SockIt! Gel™. The pH was determined periodically over a period of 24 months for the hydrogel of Example 6 and for SockIt! Gel™. The results are summarized in Table 5, and are graphically depicted in FIG. 8.

TABLE 5
pH over Time

| | Inventive | SockIt! Gel ™ |
|---|---|---|
| Time | Composition pH | pH |
| 0 months | 8.26 | 7.09 |
| 6 months | 8.26 | 6.82 |
| 12 months | 8.30 | 6.75 |
| 24 months | 8.29 | 6.5 |

The data demonstrate the hydrogel of the invention is considerably more stable than a comparable hydrogel, as shown by significantly greater pH stability or resistance to change in pH over time of the hydrogel of the invention relative to SockIt! Gel™.

### Example 8

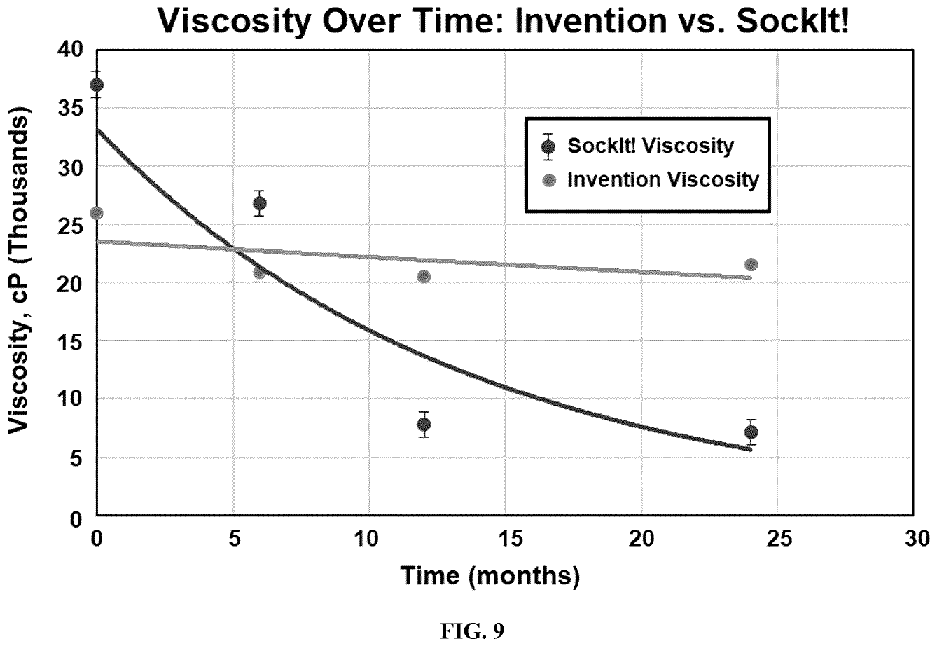

This example demonstrates the stability of an exemplary hydrogel of the invention relative to comparable hydrogel, SockIt! Gel™. The viscosity was determined periodically for the hydrogel of Example 6 and for SockIt! Gel™ over a period of 24 months. The results are summarized below in Table 6, and are graphically depicted in FIG. 9.

TABLE 6
Viscosity over Time

| | Viscosity (cP) | |
|---|---|---|
| Time | Inventive Composition | SockIt! Gel ™ |
| 0 months | 25917 | 37000 |
| 6 months | 20883 | 26750 |
| 12 months | 20500 | 7819 |
| 24 months | 21550 | 7083 |

The data demonstrate the hydrogel of the invention is considerably more stable than a comparable hydrogel as shown by significantly greater resistance to decline in viscosity over time of the hydrogel of the invention relative to SockIt! Gel™.

### Example 9

This example demonstrates the antimicrobial properties of an exemplary hydrogel of the invention. The total aerobic microbial count, total mold and yeast count, and screening for Staphylococcus aureus and Pseudomonas aeruginosa were determined over a period of 24 months for the hydrogel of Example 6. The results are summarized in Table 7.

TABLE 7
Antimicrobial Properties

| Time (months) | 0 | 6 | 12 | 24 | Specification |
|---|---|---|---|---|---|
| Total Aerobic | 30 | 2 | 1 | <1 | <200 |
| Microbial | | | | | |
| Count (CFU/g) | | | | | |
| Total Molds | 12 | <1 | <1 | <1 | <20 |
| & Yeasts | | | | | |
| Count (CFU/g) | | | | | |
| Screening for | Absent | Absent | Absent | Absent | Absent |
| S. aureus | | | | | |
| Screening for | Absent | Absent | Absent | Absent | Absent |
| P. aeruginosa | | | | | |

The results demonstrate that the hydrogel of the invention exhibits excellent resistance to microbial contamination. After 24 months, the hydrogel of the invention exhibited a total aerobic microbial count of <1 CFU/g, and a total molds and yeasts count of <1 CFU/g, falling considerably below the maximum tolerable levels required by the specification, and showed no evidence of S. aureus or P. aeruginosa as required by the specification.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. The present invention also contemplates and provides compositions that “consist essentially of” and/or that “consist of” the combinations of components described herein, as well as methods that “consist essentially of” and/or that “consist of” the combinations of method steps described herein, as the transitional phrases “consists essentially of” and “consists of” are interpreted under U.S. patent law.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A composition in the form of a hydrogel for treating wounds in the oral cavity, the hydrogel comprising one or more polysaccharides, one or more polyols, one or more essential oils, and water, wherein the hydrogel has a pH of about 6.2 to about 8.5.

2. The composition of claim 1, wherein the hydrogel has a pH of about 7.0 to about 8.5.

3. The composition of claim 1, wherein the hydrogel further comprises a bicarbonate salt.

4. The composition of claim 3, wherein the bicarbonate salt comprises lithium bicarbonate, sodium bicarbonate, potassium bicarbonate, or a combination thereof.

5. The composition of claim 3, wherein the hydrogel comprises from about 0.1 wt. % to about 2 wt. % of the bicarbonate salt.

6. The composition of claim 3, wherein the hydrogel comprises from about 0.1 wt. % to about 1 wt. % of the bicarbonate salt.

7. The composition of claim 1, wherein the one or more polysaccharides comprises guar gum, microcrystalline cellulose, carboxymethylcellulose, locust bean flour, waxy maize starch, alginate, guar flour, carrageenan, acemannan, karaya gum, gellan gum, konjac glucomannan, galactomannan, tara stone flour, propylene glycol alginate, pectin, sodium hyaluronate, tragacanth, tara gum, welan gum, agar, xanthan gum, or a combination thereof.

8. The composition of claim 1, wherein the hydrogel comprises from about 0.1 wt. % to about 5 wt. % of the one or more polysaccharides.

9. The composition of claim 1, wherein the hydrogel comprises from about 1 wt. % to about 5 wt. % of the one or more polysaccharides.

10. The composition of claim 1, wherein the one or more polyols comprises erythritol, threitol, xylitol, mannitol, sorbitol, or a combination thereof.

11. The composition of claim 1, wherein the hydrogel comprises from about 0.1 wt. % to about 5 wt. % of the one or more polyols.

12. The composition of claim 1, wherein the hydrogel comprises from about 1 wt. % to about 5 wt. % of the one or more polyols.

13. The composition of claim 1, wherein the one or more essential oils comprises anise oil, bergamot oil, cinnamon oil, clove oil, eucalyptus oil, ginger oil, lavender oil, peppermint oil, rosemary oil, spearmint, thyme oil, wintergreen oil, or a combination thereof.

14. The composition of claim 1, wherein the hydrogel comprises from about 0.05 wt. % to about 0.5 wt. % of the one or more essential oils.

15. The composition of claim 1, wherein the hydrogel comprises from about 90 wt. % to about 95 wt. % water.

16. The composition of claim 1, wherein the hydrogel comprises from about 92 wt. % to about 95 wt. % water.

17. The composition of claim 1, wherein after 24 months of storage at 25±2° C., the hydrogel exhibits less than about 50% reduction in viscosity as measured by a calibrated rotary viscometer having a rotor size of 4 and a rotary speed of 12 RPM.

18. The composition of claim 1, the pH of the hydrogel exhibits does not change by more than about 0.5 pH units after 24 months of storage at 25±2° C.

19. The composition of claim 1, wherein the hydrogel has a total aerobic microbial count of less than 5 CFU/g after 24 months of storage at 25±2° C.

20. A method for treating a wound in the oral cavity, the method comprising topically applying an effective amount of the composition of claim 1 to the wound in the oral cavity, to treat the wound in the oral cavity.

21. The method of claim 20, wherein the method comprises alleviating pain associated with the wound in the oral cavity.

22. The method of claim 20, wherein the method promotes healing of the wound in the oral cavity.
